(19) United States
(12) Patent Application Publication
(10) Pub. No.: US 2016/0000510 A1
(43) Pub. Date: Jan. 7, 2016

(54) Powered Stereotactic Positioning Guide Apparatus

(71) Applicant: Choon Kee Lee, Denver, CO (US)

(72) Inventor: Choon Kee Lee, Denver, CO (US)

(21) Appl. No.: 14/321,958

(22) Filed: Jul. 2, 2014

(51) Int. Cl. A61B 19/00; A61B 8/00; A61B 8/08

(52) U.S. Cl. CPC A61B 19/201; A61B 8/0841; A61B 8/54; A61B 8/56; A61B 8/4494; A61B 8/4455

(57) ABSTRACT

The present invention presents an apparatus and methods to stereotactically guide insertion of invasive tubular devices to a tissue target of a living body. The apparatus comprises a powered positioning guide control assembly and a positioning guide assembly that is coupled with and operated by the powered positioning guide control assembly, and rotationally adjustable and lockable. The powered positioning guide control assembly encloses an ultrasound transducer to visualize and aim at the tissue target, and adjusts an insertion angle of an invasive tubular device placed in the positioning guide assembly.

### CROSS REFERENCE TO RELATED APPLICATIONS

Attached please refer to the Information Disclosure Statement for the cross reference to related applications.

### STATEMENT REGARDING FEDERALLY SPONSORED RESEARCH OR DEVELOPMENT

The present invention is not a federally sponsored research or development.

### TECHNICAL FIELD

The present invention relates generally to the field of positioning guidance of insertion of invasive devices in a living body for medical purposes. More specifically, the present invention provides a powered apparatus and methods to guide introduction of tubular devices into a tissue using ultrasound.

### BACKGROUND OF THE INVENTION

An invasive tubular device can be guided under ultrasonographic visualization by a powered apparatus that measures an insertion angle and a depth to reach a tissue target. Insertion angle of an invasive device can also be adjusted to various positions of an ultrasound transducer in relation to a center of the tissue target. The powered apparatus comprises a positioning guide for an invasive tubular device through which the invasive tubular device passes toward a tissue target and a powered positioning guide controller which adjusts angulation of the positioning guide by using ultrasonographic visual information of a set of insertion angle and depth of the invasive tubular device to reach the tissue target. The positioning guide is configured to be coupled with the powered positioning guide controller in a way to separate the positioning guide, before inserting the invasive tubular device toward the tissue target, from the powered positioning guide controller following localization and angulation of the positioning guide. It would be technically advantageous for a majority of applications to have a free-standing positioning guide that would verify a correct positioning of the positioning guide before inserting invasive tubular devices by additional imaging modalities such as computerized tomogram or by repeating ultrasonogram. The free-standing positioning guide reversibly attached to a skin overlying the tissue target allows a few invasive tubular devices to be used interchangeably through the same positioning guide toward the same tissue target. Attachment of the free-standing positioning guide to the skin overlying the target frees an operator to use both hands for a series of procedures for manipulating these devices, without a need to hold the ultrasound probe by one hand.

There are other applications of the positioning guide, however, which would best be accomplished by an apparatus of a positioning guide coupled with a powered positioning guide controller throughout manipulations of invasive devices. In-process visualization of insertion procedures of invasive devices would be required for small lesions, lesions located near vital structures or lesions that move during invasive procedures by physiologic bodily function such as breathing, heartbeat or pulsating blood vessels to increase accuracy of the insertion and to reduce chances of potential complications of the procedure. An invasive device placed in the positioning guide can be visualized and monitored by the positioning guide controller which houses an ultrasound probe and holds the positioning guide it controls for insertion angle and depth. Multiple samplings from a few individual sites in a single lesion can be expedited by a coupled apparatus as all interested sites are visualized by a positioning guide controller in an ultrasonographic field and the positioning guide controller holding a positioning guide can select preferred sites for a series of sequential invasive procedures. One crucial advantage of using the coupled configuration of the apparatus over a free-standing positioning guide comes from a need to abort or change an invasive procedure after the procedure was initiated. There would be several reasons to abort or change invasive procedures even after an invasive device was inserted into a tissue, including an unexpected heterogeneity in consistency of the tissue that forces changes in an insertion path, a wrong insertion path that leads the invasive device to an area off a tissue target or an incidental damage to vital structures such as blood vessels. In these circumstances, a free-standing positioning guide once deployed to a skin region by a positioning guide controller will be wasted. In contrast, a positioning guide yet attached to and controlled by a powered positioning guide controller will be able to function until completion of an intended procedure.

### SUMMARY OF THE INVENTION

The present invention provides a powered apparatus that minimizes double-hand operations for guiding insertion of invasive tubular devices to tissue with ultrasonographically visualized targeting approaches to a tissue target. The invention provides a means to rotationally adjust insertion angle of invasive tubular devices to reach the tissue target, which can be monitored in an ultrasonographic field. The apparatus comprises a powered positioning guide control assembly and a positioning guide assembly that is coupled with and operated by the positioning guide control assembly. The positioning guide control assembly is configured to enclose an ultrasound transducer and coordinate adjusting an insertion angle of an invasive device by a powered motor assembly with arranging a linear alignment between a point of the transducer head and the tissue target in an ultrasonographic field.

In one embodiment, the positioning guide assembly is provided in one or a plurality of configurations, including a cross configuration which comprises an upright tubular positioning guide and a pair of transverse cylinders irreversibly attached at a right angle to each opposite side of a lower portion of an outer wall of the tubular positioning guide, respectively. One transverse cylinder is configured as a worm gear and serves for rotation of the tubular positioning guide and the other transverse cylinder provides the tubular positioning guide with rotational stability. The transverse cylinder for stability is slidably and rotatably housed in a cylinder overtube that is attached to a base panel located below said transverse cylinder.

In one embodiment, a cylinder overtube for a stabilizer cylinder of the tubular positioning guide has a horizontal slot for a length to accommodate a part of a lock and release lever which snaps in and out of said horizontal slot. An inner wall of the stabilizer cylinder overtube has a plurality of substantially linear threads. In between of an outer circumferential wall of the stabilizer cylinder and the inner wall of the stabilizer cylinder overtube, a thin nonslip tubular elastomer is provided, encasing the outer wall of said stabilizer cylinder. The horizontal slot of the stabilizer cylinder overtube is reversibly and circumferentially expandable to a degree upon engagement with the lock and release lever, which widens an inner tubular space of said stabilizer cylinder overtube. Widening of the inner tubular space allows friction-less rotation of both the elastomer and stabilizer cylinder inside said stabilizer cylinder overtube. Disengagement of the lock and release lever shrinks a circumference and the inner tubular space of said stabilizer cylinder overtube, which then holds fast both the tubular elastomer and stabilizer cylinder together. The stabilizer cylinder is fastened by friction generated by the circumferentially squeezed tubular elastomer encasing said stabilizer cylinder.

In one embodiment, the transverse cylinder of the tubular positioning guide configured as a worm gear meshes with a worm at a right angle to form a worm drive. The worm is configured to be longitudinally connected to an output shaft of a gearbox arrangement that is controllably driven by an electric motor. A proximal end of the worm is reversibly secured for axial rotation in a flange constructed on an upper surface of a base panel of the tubular positioning guide below the worm gear. A mid portion of the base panel is configured to provide an open space through which an invasive device passes from the tubular positioning guide to a tissue target.

In one embodiment, the positioning guide assembly is configured to be reversibly coupled with the positioning guide control assembly by a snap-fit insertion of a pair of vertical plates to a pair of corresponding notches on both lateral sides of a proximal upper portion of the positioning guide control assembly. An axial center of a lateral side of the worm gear is rotatably inserted to a flange located on an inner surface of one of the pair of the vertical plates, which provides rotational axis for the worm gear. A mid portion of the other vertical plate provides a rectangularly open space in which the lock and release lever is movably held along a pivoting center of said lock and release lever. Snap-fit insertion of the lock and release lever into the corresponding notch on the lateral side of the proximal upper portion of the positioning guide control assembly is coincided with engagement of said lever with the horizontal slot of the stabilizer cylinder overtube, which results in widening of the inner tubular space of said stabilizer cylinder overtube. Retracting said lock and release lever from said notch of the positioning guide control assembly disengages said lever from the horizontal slot of the stabilizer cylinder overtube. Whether the lock and release lever is engaged with or disengaged from the horizontal slot of the stabilizer cylinder overtube, the positioning guide assembly stays coupled with the positioning guide control assembly by the vertical plates inserted into the pair of corresponding notches of said positioning guide control assembly.

In one embodiment, the positioning guide control assembly is provided in one or a plurality of configurations including a modular configuration which comprises a transducer enclosure, a position alignment assembly, a powered positioning control assembly, a gear output shaft enclosure, a power and electronic control assembly and a handle assembly. The transducer enclosure is provided in a closed longitudinal box configuration with its proximal portion open to allow a face portion of a transducer to contact a distal part of the position alignment assembly via a solid gel panel. Proximal to the position alignment assembly, the transducer enclosure provides an open rectangular space to accommodate a second solid gel panel that contacts a skin overlying a tissue target. The transducer enclosure is configured to enclose the transducer in a manner to align longitudinal and horizontal axes of the transducer in parallel with longitudinal and horizontal axes of said transducer enclosure, respectively. Both the horizontal and longitudinal axes of the transducer are used as reference axes to calibrate angular displacement of the tubular positioning guide. A bottom portion of a distal portion of the transducer enclosure opens to the handle assembly through which electric cables pass. A distal end of the transducer enclosure adjoins a compartment for the powered positioning control assembly.

In one embodiment, the position alignment assembly is provided in one or a plurality of electromechanical configurations, which comprises a substantially ultrasound-transparent flat rectangular box and an electromagnetic pointing device. The flat rectangular box is configured as leakproof, is filled with an ultrasound-transparent liquid which is electrically non-conductive. The flat rectangular box is located proximal to the face of the transducer. In one example, the position alignment assembly comprises a galvanometer-type electromagnetic pointing device that uses varying electric voltage, current or resistance to radially move a linear movable pointer around a center of said device. The linear movable pointer is configured to block ultrasound transmission, which is visualized in an ultrasonographic view.

In one embodiment, the positioning control assembly is provided in one or a plurality of configurations including a rectangular box configuration which encloses an electric motor, a gearbox and a rotary position sensing device such as potentiometer, optical encoder or magnetic encoder. The electric motor is irreversibly fixed to a distal wall of the positioning control assembly, with its rotor protruding longitudinally along the axis. A protruded portion of the rotor is configured as a longitudinal spur gear that meshes in parallel with a cylindrical spur gear. The cylindrical spur gear is connected to the position sensing device coaxially that measures rotational displacements of said cylindrical spur gear. The position sensing device is electronically connected to the power and electronic control assembly that relays an electronic information from said position sensing device of rotational displacements of the cylindrical spur gear to the electromagnetic pointing device of the position alignment assembly. The cylindrical spur gear meshes with another longitudinal spur gear that coaxially merges with the output shaft located outside the positioning control assembly. The output shaft is provided in one or a plurality of configurations and is housed in the gear output shaft enclosure. A proximal end of the output shaft protrudes from an opening located at a proximal end of the output shaft enclosure and is configured to be reversibly connected to a distal end of the worm. A switch located on an outer surface of the handle assembly is electrically connected to the power and electronic control assembly and to the positioning control assembly and is configured to turn on for a controllably variable duration and off the electric motor. Rotations of the electric motor are transmitted to the output shaft that in turn rotates the worm of the worm drive arrangement for the positioning guide assembly.

In one embodiment, the gear output shaft enclosure is provided in one or a plurality of configurations including a longitudinal tubular structure located on an upper surface of both the positioning control assembly and transducer enclosure. The output shaft enclosure has a proximal end having an opening through which the output shaft protrudes and a distal end which provides a central tubular cup to accommodate a distal end of the output shaft for axial rotation. The output shaft enclosure is configured to provide a means to reduce rotational friction between the output shaft and the output shaft enclosure, which includes a portion having a rolling-element bearing.

In one embodiment, the power and electronic control assembly is provided in one or a plurality of configurations including a rectangular box configuration which has an integrated circuit board, a segment digital display, a control knob connected to the integrated circuit board and a power source. The integrated circuit board is located distally and electronically connected to the segment digital display, the positioning control assembly, the position alignment assembly and the switch of the handle assembly. In one configuration, a compartment for replaceable batteries is located inside the positioning control assembly and connects batteries electrically with the integrated circuit board, the segment digital display, the positioning control assembly, the position alignment assembly and the switch of the handle assembly. The electronic control assembly is located distal to the positioning control assembly and the segment digital display is configured to be visible on a distal outer surface of the integrated circuit board. The segment digital display shows at least a digitized numerical information about a distance between a position of the linear movable pointer tangentially placed over the tissue target and said tissue target.

In another embodiment, the power and electronic control assembly is configured to control movement of the electromagnetic pointing device of the position alignment assembly upon an electronic input from the position sensing device. In this configuration, rotation of the worm gear of the positioning guide assembly by the electric motor of the positioning control assembly translates into ultrasonographically visualizable movement of the linear movable pointer of the electromagnetic pointing device of the position alignment assembly. In a two-dimensional ultrasonographic view, the linear movable pointer is configured to produce a thin vertically linear shadow line that can be distinguished readily from surrounding tissue images. Rotation of said worm gear is configured to match horizontal movement of said linear movable pointer in ways that a longitudinal axis of an invasive device at an insertion angle in the positioning guide assembly crosses a linear shadow line at a center of a tissue target in the two-dimensional ultrasonographic view.

### BRIEF DESCRIPTION OF THE DRAWINGS

FIG. 1 shows a schematic illustration of an example of a positioning guide assembly (A) and a positioning guide control assembly (B).

FIG. 2 shows a schematic example of individual components of the positioning guide assembly of the apparatus: FIG. 2A represents a positioning guide assembly and a lock and release lever shown separately for illustration; FIG. 2B shows a stabilizer cylinder overtube of the positioning guide assembly; FIG. 2C shows an internal view of the positioning guide assembly; FIG. 2D shows individual components of a tubular positioning guide.

FIG. 3 shows a schematic illustration of an example of individual parts of a positioning guide assembly and a positioning guide control assembly.

FIG. 4 shows a schematic three-quarter view of coupling between a positioning guide assembly and a positioning guide control assembly.

FIG. 5 shows a schematic example of individual compartments of a positioning guide control assembly.

FIG. 6 shows a schematic example of components of a positioning control assembly of the positioning guide control assembly.

FIG. 7 shows a schematic illustration of an example of a galvanometer-type position alignment assembly.

FIG. 8 depicts a schematic illustration of components housed in the positioning guide control assembly.

FIG. 9 illustrates an schematic example of mechanisms of locking and unlocking of the tubular positioning guide; FIG. 8A shows an unlocked configuration of the tubular positioning guide; FIG. 8B shows a locked configuration; FIGS. 8C and 8D show a cross-sectional view of unlocked and locked configurations, respectively.

FIG. 10 illustrates a schematic example of a sequence of action of the present invention.

### DETAILED DESCRIPTION OF THE DRAWINGS

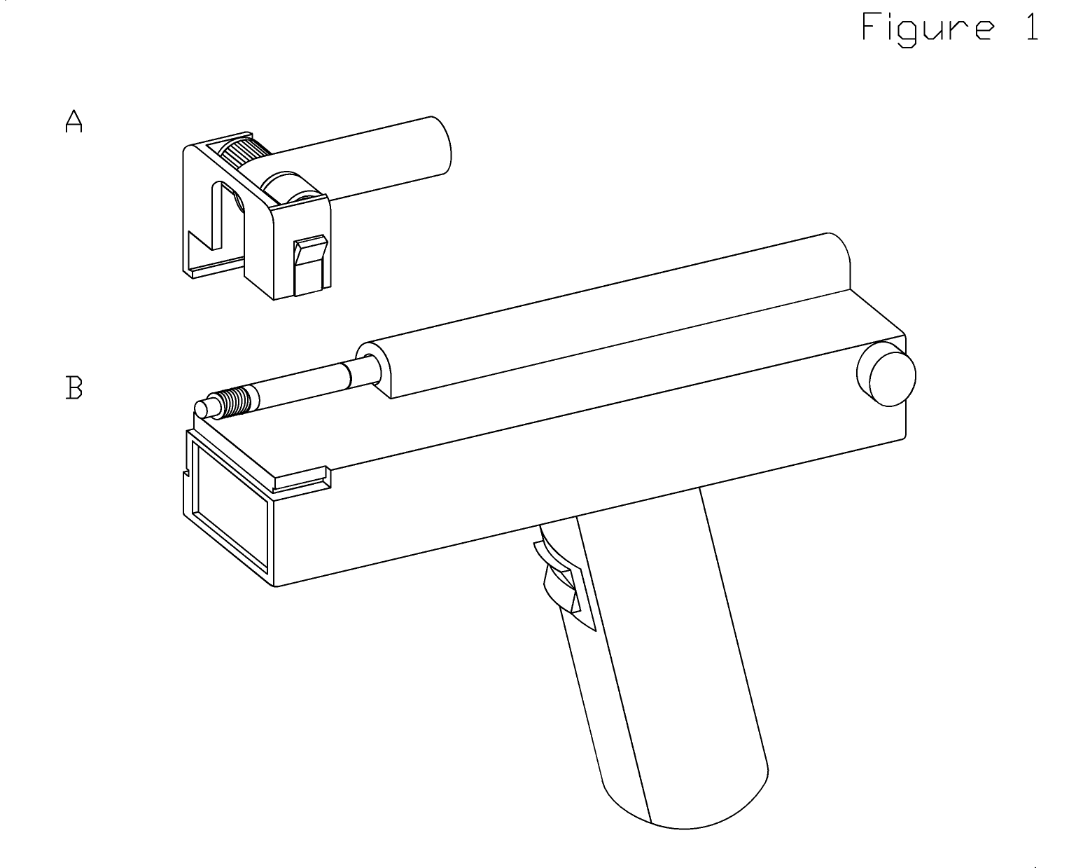

As described below, the present invention provides a powered positioning guide apparatus stereotactically aiming at a tissue target and methods of use. It is to be understood that the descriptions are solely for the purpose of illustrating the present invention, and should not be understood in any way as restrictive or limited. Embodiments of the present invention are preferably depicted with reference to FIGS. 1 to 10, however, such reference is not intended to limit the present invention in any manner. The drawings do not represent actual dimension of devices, but illustrate the principles of the present invention.

FIG. 1 shows a schematic illustration of an example of a positioning guide assembly and a positioning guide control assembly. FIG. 1A represents the positioning guide assembly and FIG. 1B represents the positioning guide control assembly. The positioning guide assembly is configured to reversibly be coupled with the positioning guide control assembly.

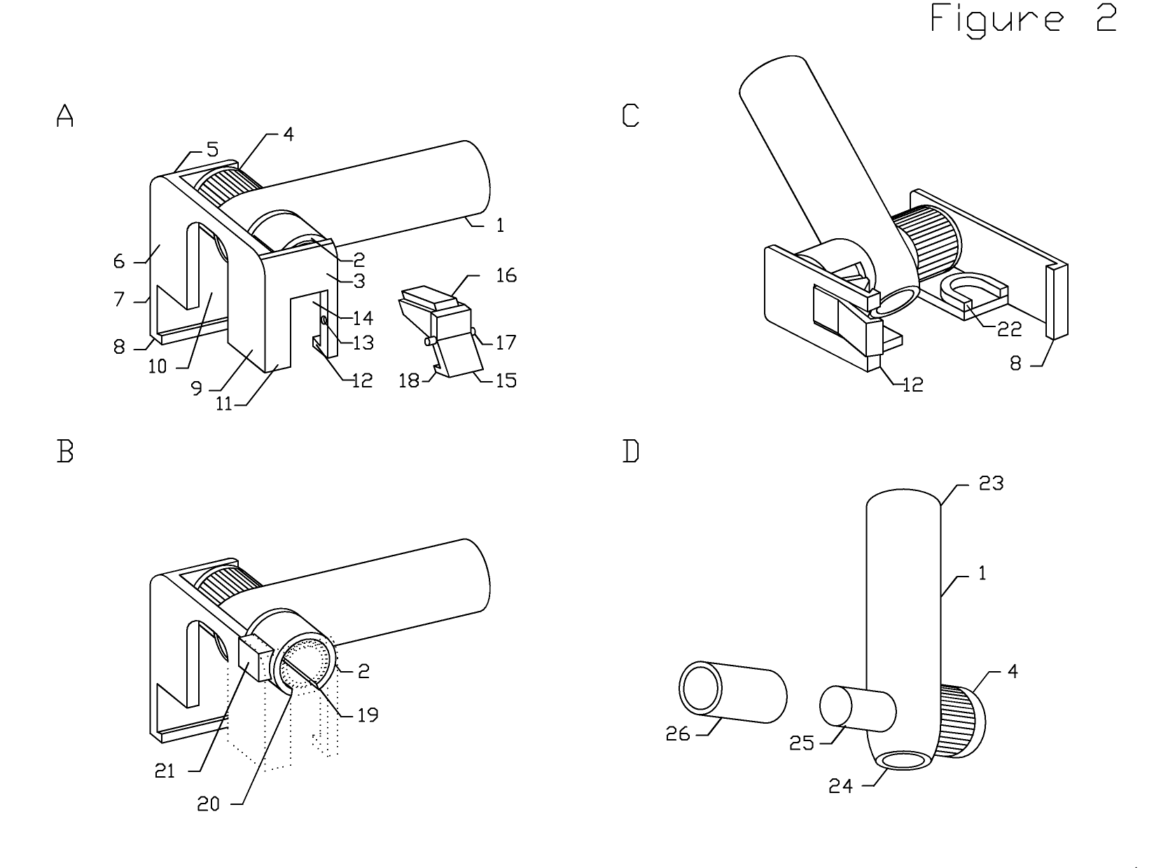

FIG. 2 shows a schematic example of individual components of the positioning guide assembly of the apparatus. FIGS. 2A and 2B show a three-dimensional view of the positioning guide assembly which comprises the tubular positioning guide 1 fixedly attached at a right angle to a worm gear 4 and slidably inserted in a stabilizer cylinder overtube 2. A pivoting center of the worm gear 4 is supported by a lateral guide plate 5. An opposite lateral guide plate 3 supports a rocker-switch-type lock and release lever 15 in a rectangularly open space 14. A top portion of the lock and release lever 15 has a substantially rectangular protuberance 16 that slides in and out of a horizontal slot located in a bottom portion of the stabilizer cylinder overtube 2. The horizontal slot is bordered by a pair of lateral edges 19 and 20 of the stabilizer cylinder overtube 2. The lock and release lever 15 pivots about a pin 17 that is slidably inserted in a pair of recesses, with one of which shown as 13. The lock and release lever 15 has a linear snap-fit edge 18 that reversibly fastens said lock and release lever to a positioning guide control assembly. Both the lateral guide plates 3 and 5 adjoin a pair of bottom panels 9 and 6 at a right angle, respectively. The lateral guide plate 5 extends to a flat portion 7 that is distally bordered by a linear snap-fit edge 8. Similarly, a distal end 11 of the lateral guide plate 3 is bordered by a pair of linear snap-fit edges 12 on both sides of the rectangularly open space 14. Both the snap-fit edges 8 and 12 are configured to be reversibly coupled with a pair of corresponding notches of the positioning guide control assembly. In between of both the bottom panels 6 and 9, an open space 10 is provided through which devices pass toward a tissue target. The stabilizer cylinder overtube 2 is irreversibly attached to the bottom panel 9 via a supporting block 21.

FIG. 2C shows edges 8 and 12 of the lateral guide plates 5 and 3, respectively. There is provided a worm shaft holder 22 that reversibly secures a proximal end of a worm shaft during rotation of the worm gear 4. FIG. 2D shows individual components of the tubular positioning guide 1 with a tip 24 and a top portion 23 through which devices pass. The worm gear 4 is attached to one lateral side of an outer wall of the tubular positioning guide and a stabilizer cylinder 25 is attached to an opposite side of the outer wall. Both are fixedly attached to the outer wall. The stabilizer cylinder 25 stabilizes the tubular positioning guide during rotation and is slidably encased by a thin non-slip tubular elastomer 26. The tubular elastomer 26 is located in between of an outer wall of the stabilizer cylinder 25 and an inner wall of the stabilizer cylinder overtube 2 and provides friction on both the walls. The positioning guide assembly is non-reusable.

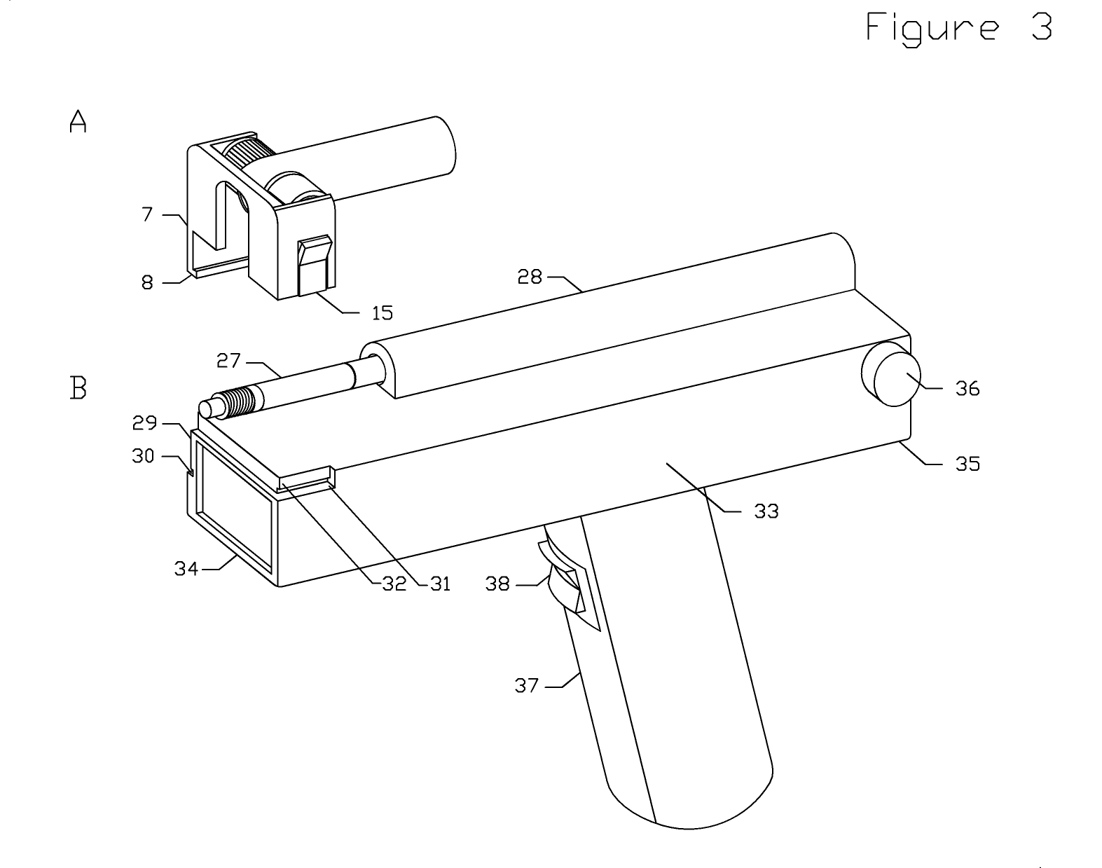

FIG. 3 shows a schematic illustration of an example of a positioning guide control assembly. The positioning guide control assembly is provided in one or a plurality of configurations including a longitudinal box configuration with a handle attached to a bottom of said guide control assembly. As shown in FIG. 3B, the positioning guide control assembly comprises a transducer enclosure 33 that anteriorly adjoins a proximal portion 34 and posteriorly a distal portion 35. A handle assembly 37 is attached to a lower wall of the transducer enclosure 33. Both the transducer enclosure 33 and handle assembly 37 house an ultrasound transducer and electric cables, which are connected to a main ultrasonographic machine. Control of the positioning guide control assembly is accomplished by an electric switch 38 located on an anterior part of the handle assembly 37. A pair of notches 30 and 31 are provided for a length on both upper lateral sides of the proximal portion of the positioning guide control assembly shown in FIG. 3B, which is configured to reversibly be coupled with the corresponding pair of the edges 8 and 12, respectively, of the positioning guide assembly shown in FIG. 3A. Upper borders of the notches 30 and 31 vertically adjoin recessed portions 29 and 32 of said upper lateral sides of said proximal portion, which anchor the edges 8 and 12. The proximal portion 34 provides space for solid gel panels and a position alignment assembly and the distal portion 35 houses a power and electronic control assembly. A control knob 36 is located on one lateral wall of the distal portion 35, which is connected to the power and electronic control assembly and provides said power and electronic control assembly with a range of numerical information. An output shaft enclosure 28 is attached longitudinally to a part of an upper surface of the transducer enclosure 33. A worm shaft 27 is reversibly connected to a gear shaft protruding from the output shaft enclosure 28.

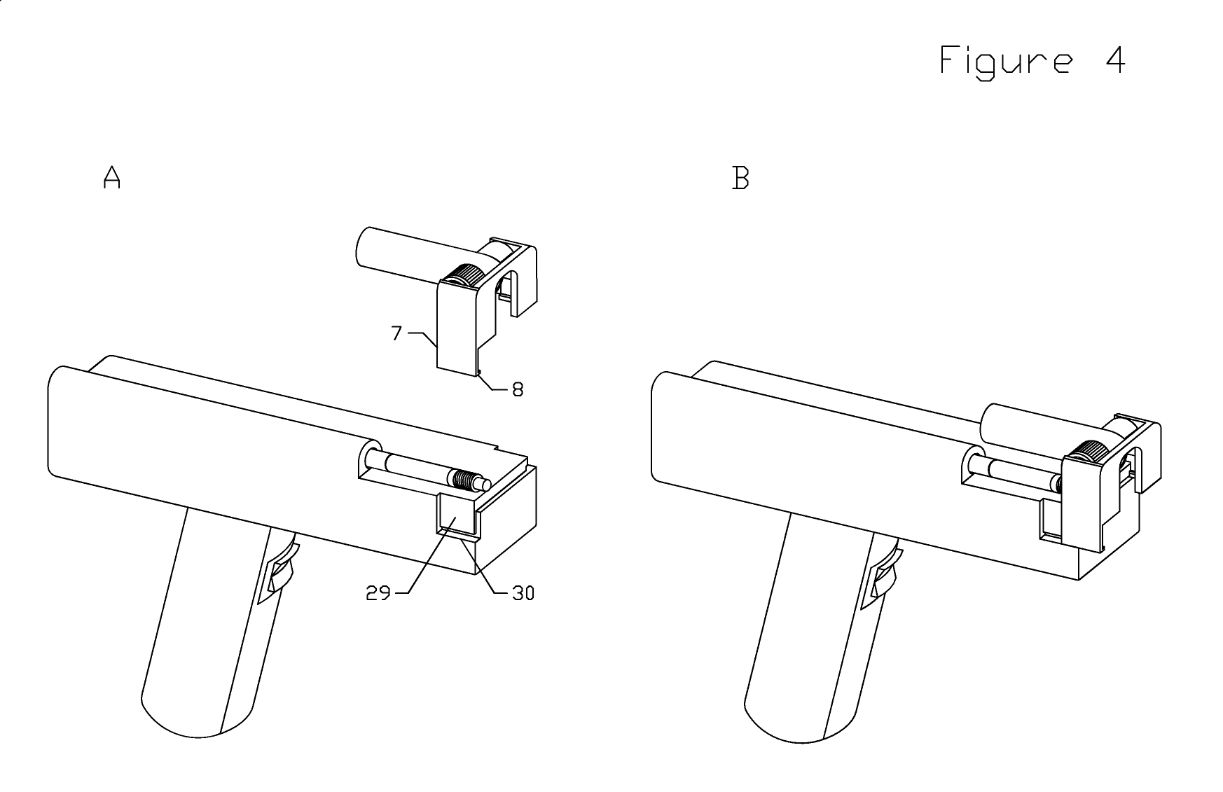

FIG. 4 shows a schematic example of coupling between a positioning guide assembly and a positioning guide control assembly. Shown in FIG. 4A, the edge 8 of the lateral guide plate is coupled with the notch 30 by a snap-fit insertion, which makes an outer surface of the recessed portion 29 come in contact with an inner surface of the flat portion 7 of the lateral guide plate. Coupling of the edge 8 with the notch 30 prevents disengagement of the positioning guide assembly from the positioning guide control assembly at a right angle to the longitudinal axis of the transducer housing. The positioning guide control assembly, however, is configured with an open proximal end of the notch to let the positioning guide assembly proximally slide out along the longitudinal axis of said transducer housing, as shown in FIG. 4B. There is provided a contact surface between the flat portion 7 and the recessed portion 29, which allows said positioning guide assembly to be securely coupled with and removed from said positioning guide control assembly. Referring to FIGS. 2A and 2C, an engaged flat portion 7 with the recessed portion 29 also assists the worm shaft holder 22 in securing a proximal end of a worm shaft during rotation of the worm gear 4.

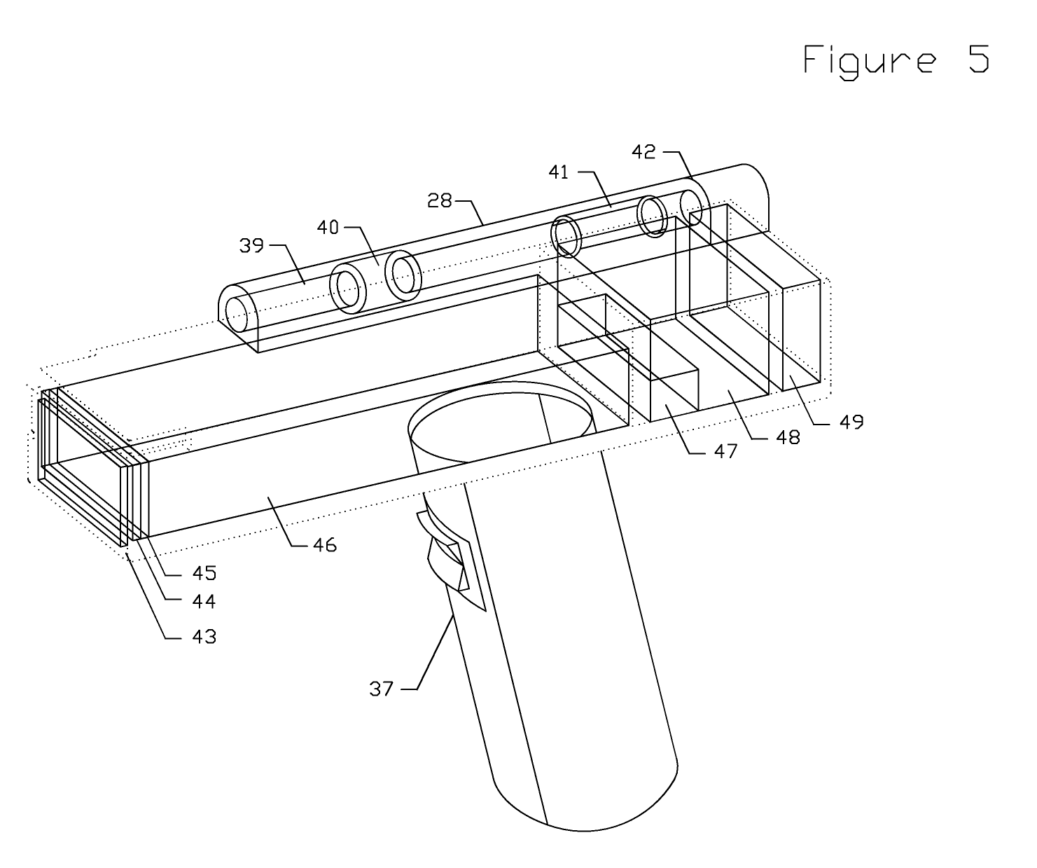

FIG. 5 shows a schematic see-through illustration of an example of individual compartments of the positioning guide control assembly. The proximal portion of the positioning guide control assembly is provided in one or a plurality of configurations, including rectangularly tubular compartments 43 and 45 to reversibly hold a pair of solid gel panels to enhance ultrasound transmission between a face of the transducer and a tissue, and another rectangularly tubular compartment 44 located in between of the spaces 43 and 45 to house a position alignment assembly. Distal to the compartment 45, there is provided a rectangularly tubular space for a compartment 46 for the transducer, a battery compartment 47, a compartment 48 for a gearbox of a positioning control assembly and a compartment 49 for an electronic control assembly. A lower wall of the transducer compartment 46 adjoins an open upper part of the tubular handle assembly 37. The output shaft enclosure 28 is provided in one or a plurality of tubular configurations, which comprises an output shaft housing 39, a housing 40 for a rolling-element bearing portion of the output shaft, an output shaft gear housing 41 and a distal portion 42. A bottom of the output shaft gear housing 41 is open to an upper part of the gearbox compartment 48 to allow meshing of the shaft gear with a gear of the gearbox.

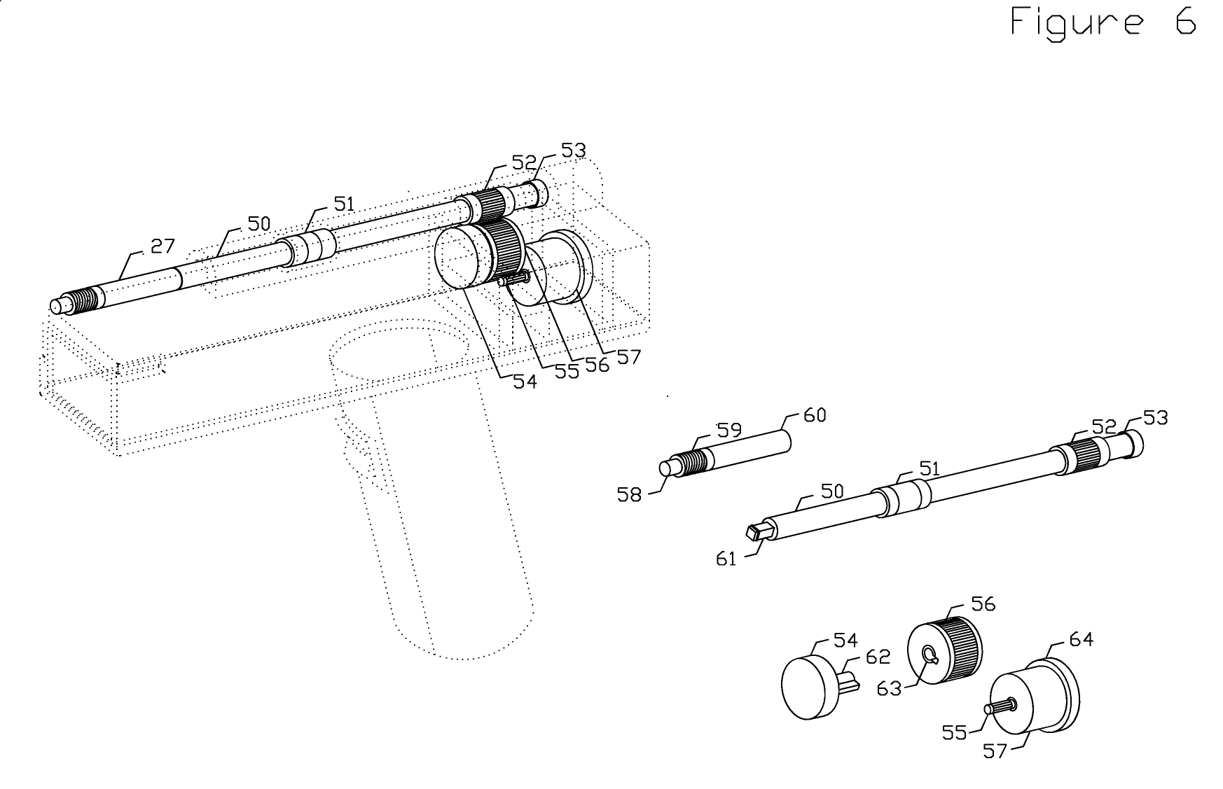

FIG. 6 shows a schematic example of a positioning control assembly, provided in one or a plurality of configurations including a parallel spur gear arrangement, which comprises an electric motor 57, a pair of spur gears 56 and 52, and a position sensing device 54. The electric motor 57 is irreversibly fastened by a flange 64 to a distal wall of the positioning control assembly, with its rotor 55 protruding longitudinally along the axis. A protruded portion of the rotor 55 is configured as a longitudinal spur gear that meshes in parallel with the cylindrical spur gear 56. The cylindrical spur gear 56 is connected coaxially to the position sensing device 54 by coupling of a central rotatable rod 62 of said position sensing device with a central tubular space 63 of said spur gear. The position sensing device 54 is fastened to a proximal wall of the positioning control assembly. The position sensing device 54 measures rotational displacements of said cylindrical spur gear 56 and is electronically connected to the power and electronic control assembly that relays an electronic information from said position sensing device of rotational displacements of said cylindrical spur gear 56 to the position alignment assembly. The cylindrical spur gear 56 meshes with another longitudinal spur gear 52 that merges with an output shaft 50 located inside the output shaft enclosure. The output shaft 50 is provided in one or a plurality of configurations and transfers axial rotation to the worm shaft 27. Referring to FIG. 5, a proximal end 61 of the output shaft 50 protrudes from an opening located at a proximal end of the output shaft enclosure 28 and is configured to be reversibly connected to a distal end 60 of the worm shaft 27. The worm shaft 27 comprises a proximal end 58, the worm 59 and the distal end 60 that has a longitudinal slot inside for a length to accommodate the proximal end 61 of the output shaft 50. Referring to FIG. 5, the distal portion 42 of the output shaft enclosure 27 provides a central tubular cup 53 to accommodate a distal end of the output shaft for axial rotation. Referring to FIG. 3, the switch 38 of the handle assembly 37 is electrically connected to the positioning control assembly and is configured to turn on for a controllably variable duration and off the electric motor 57. Rotations of the electric motor 57 are transmitted to the output shaft 50 that in turn rotates the worm shaft 27 of the worm drive arrangement for the positioning guide assembly. Transfer of rotational torque of the output shaft 50 to the worm shaft 27 is assisted by a rolling-element bearing arrangement 51 that is configured to reduce friction between the output shaft enclosure and the output shaft.

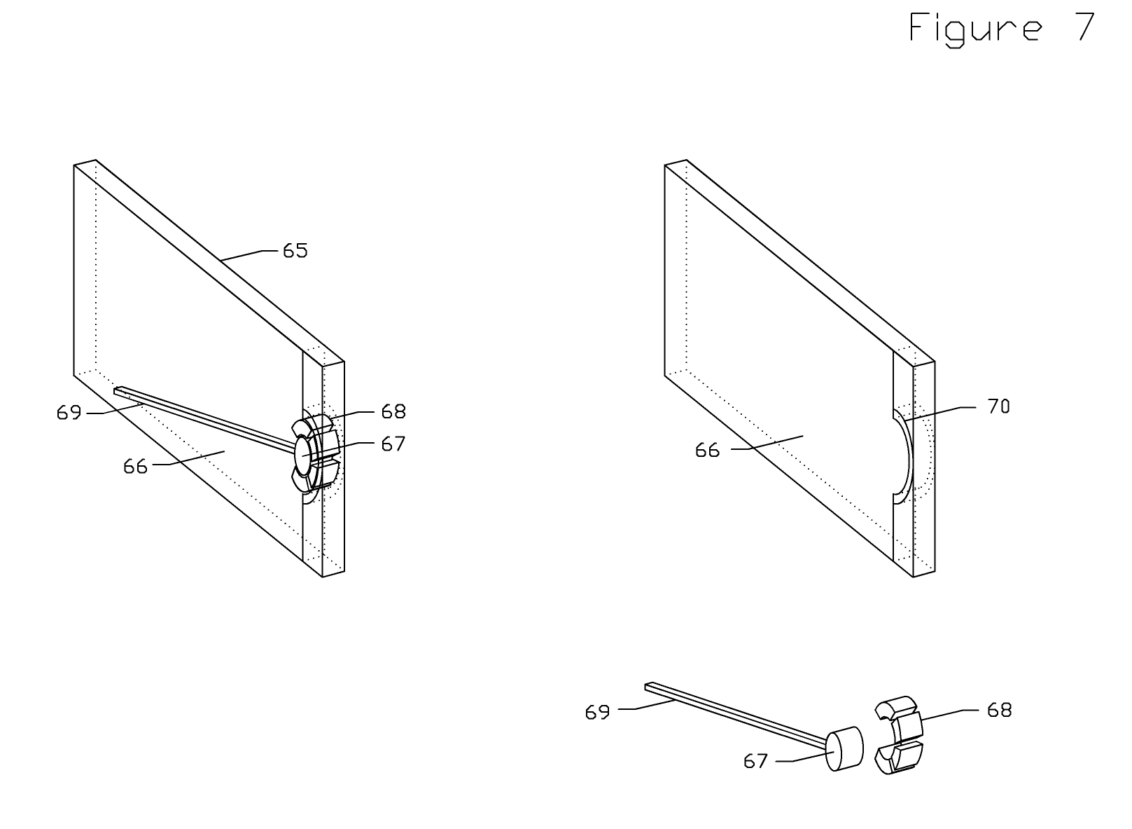

FIG. 7 shows a schematic illustration of an example of a position alignment assembly, provided in one or a plurality of electromechanical configurations, which comprises a substantially ultrasound-transparent flat rectangular box 65 and an electromagnetic pointing device 6769. The flat rectangular box 65, provided in one or a plurality of configurations, is located proximal to the face of the transducer, which is made of substantially ultrasound-transparent polymer(s), filled with one or a plurality of type(s) of substantially ultrasound-transparent liquid and leakproof. The substantially ultrasound-transparent liquid is electrically non-conductive. In one embodiment, the position alignment assembly comprises a galvanometer-type electromagnetic pointing device that uses varying range of electric voltage, current or resistance to radially move a linear movable pointer 69 around a center of said device. The linear movable pointer 69 is configured to protrude into a space 66 in the flat rectangular box 65, to move inside said flat rectangular box from side to side and to block ultrasound transmission at a right angle, which is visualized in an ultrasonographic view. The linear movable pointer 69 is configured to have a means to reduce drag upon moving inside the liquid. The galvanometer-type device comprises a U-shaped set of electromagnetic windings 68 circumferentially surrounding a pivoting wire core 67 and the linear movable pointer 69 connected to the pivoting wire core 67. A semicircular wall 70 immobilizes the windings 68 in a U-shaped configuration. Both the pivoting wire core 67 and the windings 68 are electrically connected to the power and electronic control assembly. All components of the galvanometer-type electromagnetic pointing device are configured as waterproof. Both proximal and distal surfaces of the flat rectangular box contact with a pair of the solid gel panels to enhance ultrasound transmission.

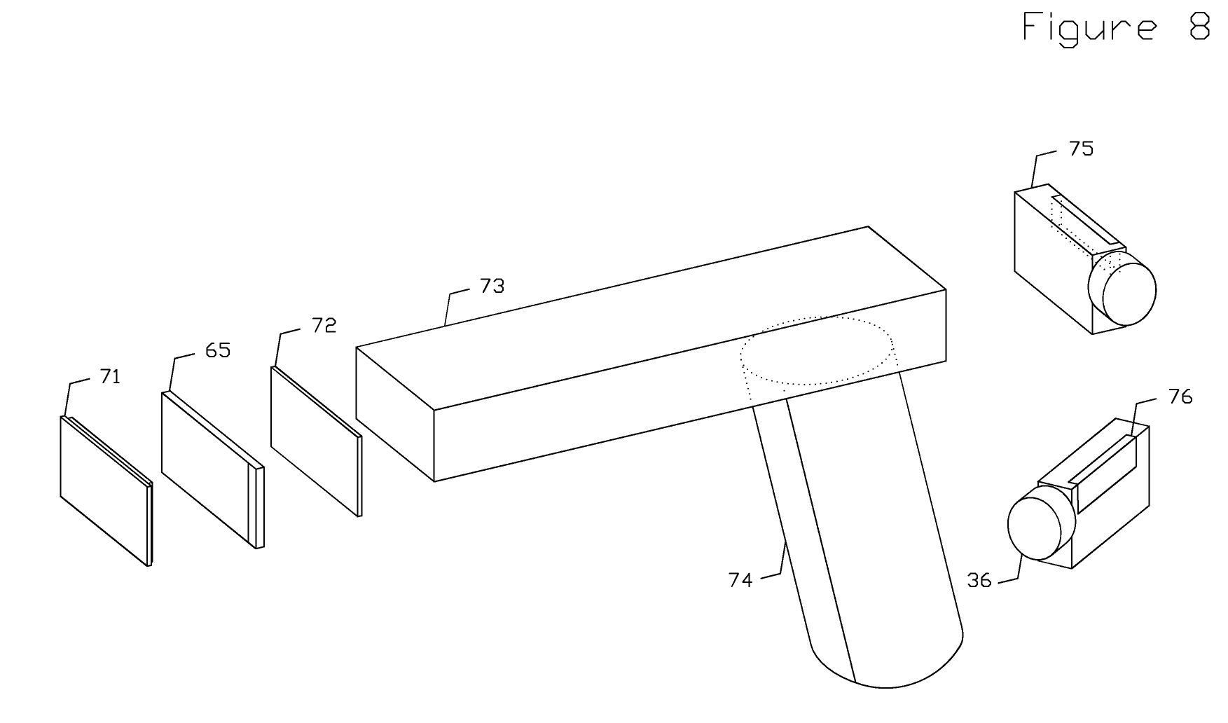

FIG. 8 depicts a schematic illustration of components housed in the positioning guide control assembly. A non-reusable solid gel panel 71 slidably is placed in front of the position alignment assembly 65 and a second solid gel panel 72 is placed in between of said position alignment assembly 65 and an ultrasound transducer 73. The solid gel panel 71 contacts with a skin overlying a tissue target. The transducer 73 is configured to be electrically connected to a main ultrasonographic machine through electric cables housed in a handle portion 74 attached to a bottom of said transducer. The electronic control assembly 75 having an integrated circuit board with a segment digital display 76 is placed in the distal portion of the positioning guide control assembly. The segment digital display 76 is configured to be seen through the distal wall of said positioning guide control assembly. The rotatable knob 36 is connected to the electronic control assembly 75, which is configured to provide the integrated circuit board with a range of numerical information.

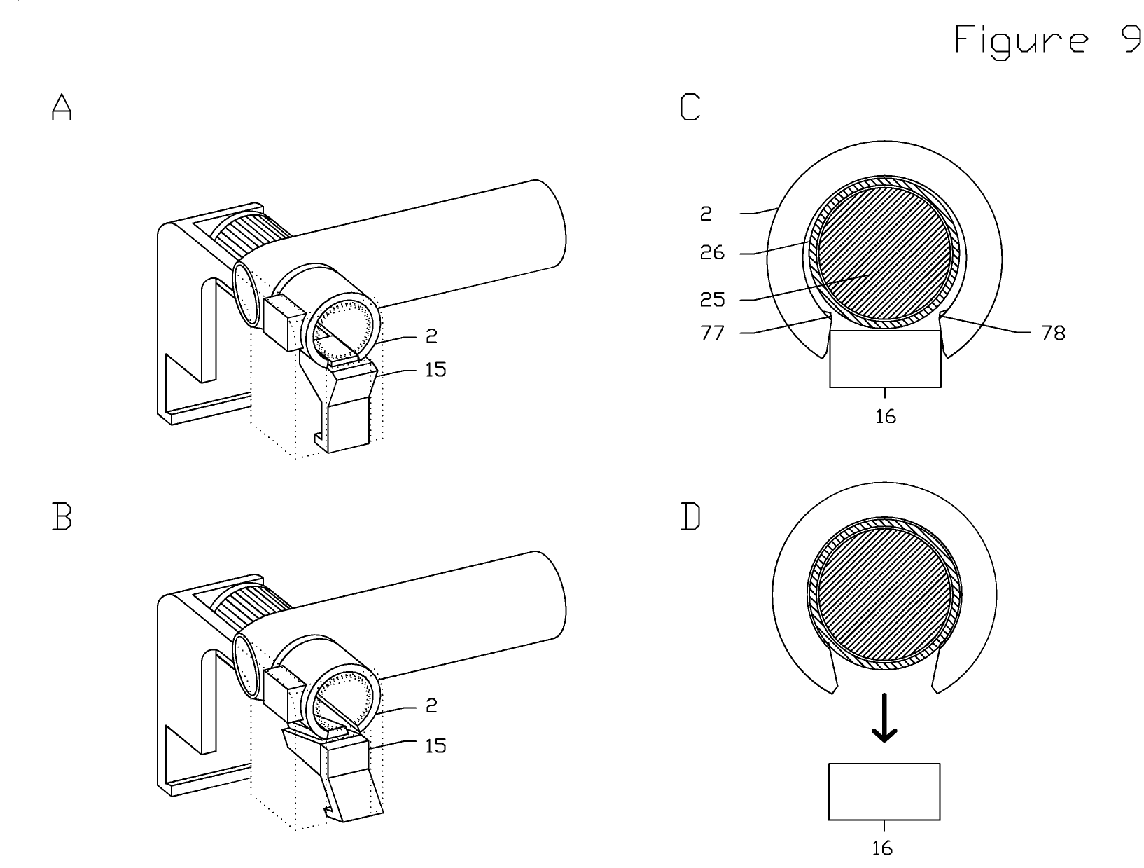

FIG. 9 illustrates an schematic example of mechanisms of locking and unlocking of the tubular positioning guide. FIGS. 9A and 9C show an unlocked configuration of the tubular positioning guide. Once the protuberance 16 of the lock and release lever 15 is inserted in the horizontal slot of the stabilizer cylinder overtube 2, it widens a circumference and an inner tubular space of the stabilizer cylinder overtube 2 and releases the non-slip elastomer 26 from a plurality of horizontally linear threads 77 and 78 located on an inner wall of the stability cylinder overtube 2. Widening of the circumference and the inner tubular space of the stabilizer cylinder overtube allow axial rotation of both the non-slip elastomer 26 and the stabilizer cylinder 25. FIGS. 8B and 8D show a locked configuration of the tubular positioning guide. The protuberance 16 of the lock and release lever 15 pivotably moves out from the horizontal slot of the stabilizer cylinder overtube 2, which allows said stabilizer cylinder overtube to circumferentially shrink and fasten both the non-slip elastomer 26 and the stabilizer cylinder 25 thereby preventing axial rotation of said elastomer and said stabilizer cylinder.

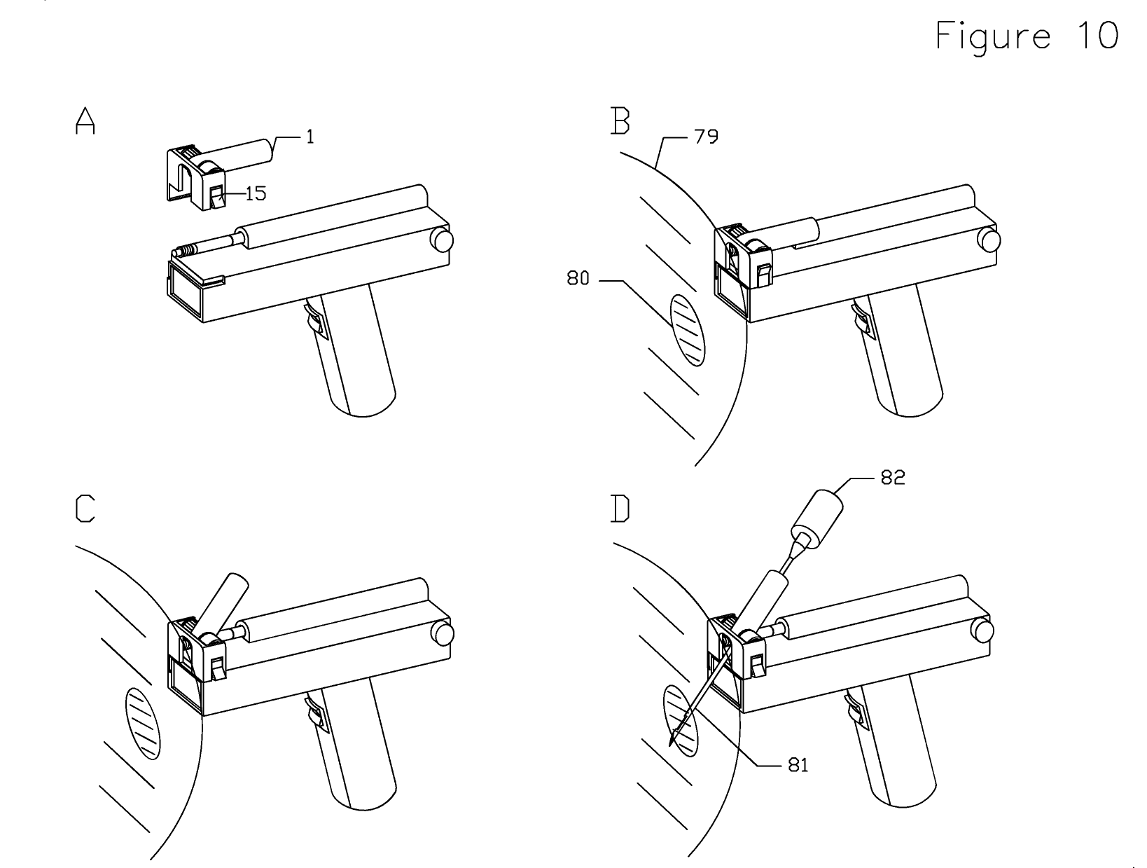

FIG. 10 illustrates a schematic example of a sequence of action of the present invention. FIG. 10A shows an uncoupled set of a positioning guide control assembly and a positioning guide assembly. In this illustration, a tubular positioning guide 1 remains fastened by a disengaged lock and release lever 15 to prevent free rotation of said tubular positioning guide inside the positioning guide assembly. Once coupled together as shown in FIG. 10B, the lock and release lever of the positioning guide assembly engages the positioning guide control assembly. An engaged lock and release lever frees the tubular positioning guide for rotation. The apparatus then contacts with a skin 79 overlying a tissue target 80. In FIG. 10C, the positioning guide control assembly rotates the tubular positioning guide to a certain angle for guiding an invasive tubular device to a tissue target. Following confirmation of an accurate angulation of the tubular positioning guide, the lock and release lever reverts back to the disengaged position that fastens the tubular positioning guide. An invasive tubular device 81 connected to a distal part 82 is then inserted into the tissue target 80 through the positioning guide assembly which maintains a contact with the skin overlying the tissue target during the procedure, as shown in FIG. 10D.

It is to be understood that the aforementioned description of the apparatus and methods is simple illustrative embodiments of the principles of the present invention. Various modifications and variations of the description of the present invention are expected to occur to those skilled in the art without departing from the spirit and scope of the present invention. Therefore the present invention is to be defined not by the aforementioned description but instead by the spirit and scope of the following claims.

What is claimed is:

1. A powered stereotactic positioning guide apparatus, comprising:
a positioning guide means, reversibly coupled with and operated by a positioning guide control means;
the positioning guide means, provided in one or a plurality of mechanical configurations, which an invasive tubular device slidably passes through, which directs the invasive tubular device in a range of insertion angles to a tissue target, which has means to rotationally adjust and reversibly lock the insertion angle of the invasive tubular device; and
the positioning guide control means, provided as one or a plurality of operating devices having one or a plurality of mechanical and electronic configurations, which has a powered means to align a longitudinal axis of the invasive tubular device in the positioning guide means with the tissue target in an ultrasonographic field, which has a means to calculate the insertion angle of the invasive tubular device housed in the positioning guide means to reach the tissue target, which measurably and controllably adjusts the insertion angle of the invasive tubular device, and which encloses an ultrasound transducer.

2. The powered stereotactic positioning guide apparatus according to claim 1, wherein the positioning guide means comprises:
a tubular positioning guide, a pivotable means, a lock and release means and a coupling means;
the tubular positioning guide, provided in one or a plurality of configurations having a conduit for invasive tubular devices, which joins the pivotable means in one or a plurality of configurations including a cross configuration, which is rotatable about a joint with the pivotable means, and which allows an invasive tubular device to pass through said conduit to reach a tissue target;
the pivotable means, provided in one or a plurality of configurations, which transmits powered rotational torque from the positioning guide control assembly to the tubular positioning guide, which pivots the tubular positioning guide about the joint with said tubular positioning guide, and which assists rotation of the tubular positioning guide;
the lock and release means, provided in one or a plurality of configurations, which reversibly fastens the tubular positioning guide and releases said tubular positioning guide for rotation, and which reversibly couples with and uncouples from the positioning guide control means; and
the coupling means, provided in one or a plurality of configurations, which reversibly couples with and uncouples from the positioning guide control means and which provides the positioning guide means with attachment to the positioning guide control means.

3. The powered stereotactic positioning guide apparatus according to claim 1, wherein the positioning guide control means comprises:
a position alignment means, a positioning control means, a power and electronic control means, an ultrasound transducer enclosure and a handle;
the position alignment means, provided as one or a plurality of operating devices having one or a plurality of mechanical and electronic configurations including an electromagnetic configuration, which electrically is connected to the positioning control means and to the power and electronic control means, which provides an ultrasonographic position information of a tissue target in relation to a position of an ultrasound transducer placed over the tissue target and which provides a means to coordinate alignment of a longitudinal axis of an invasive tubular device with said tissue target;
the positioning control means, provided as one or a plurality of powered operating devices having one or a plurality of mechanical and electronic configurations, which comprises a gearbox arrangement having a gear output means driven by an electric motor and an electronic means to measure rotational displacements of the gearbox, which electrically is connected to the position alignment means and to the power and electronic control means and which provides the tubular positioning guide of the positioning guide means with measured and controlled rotation for insertion of an invasive tubular device to a tissue target;
the power and electronic control means, provided in one or a plurality of electronic configurations, which provides the apparatus with electricity, which provides numerical calculations and data for a range of insertion angles of an invasive tubular device placed in the tubular positioning guide of the positioning guide means to reach a tissue target and which electronically coordinates the positioning control means with the position alignment means;
the transducer enclosure, provided in one or a plurality of configurations, which houses an ultrasound transducer, and which aligns with the ultrasound transducer along longitudinal and horizontal axes; and
the handle, provided in one or a plurality of configurations including a tubular configuration, which is connected to a lower wall of the transducer enclosure, which serves as a conduit for electric cables between the transducer and a main ultrasonographic machine and which has an electric switch controlling the positioning guide control means.

4. The positioning guide control means according to claim 3, wherein the position alignment means includes an electromagnetic galvanometer-type device.

5. The positioning guide control means according to claim 3, wherein the electronic means to measure rotational displacements of the gearbox includes an electronic position sensing device.

6. The positioning guide control means according to claim 4, wherein electric output of the position sensing device and movement of movable parts of the position alignment means are configured to be matched at a range of ratios.

7. A method for the powered stereotactic positioning guide apparatus according to claim 2, wherein the positioning guide means stays coupled with the positioning guide control means by the coupling means of said positioning guide means during procedure.

8. A method for the powered stereotactic positioning guide apparatus according to claim 3, wherein the positioning guide control means measurably and controllably generates and transmits powered rotation to the tubular positioning guide of the positioning guide means.

9. A method for the powered stereotactic positioning guide apparatus according to claim 3, wherein rotation of the tubular positioning guide means is measured by an electronic position sensing device.

10. A method for the powered stereotactic positioning guide apparatus according to claim 3, wherein powered rotation of the tubular positioning guide electronically controls movement of movable parts of the position alignment means.
